SEQUENCE LISTING (13 sequences)

SEQ ID NO: 1 - protein, 575 aa, HOMO SAPIENS
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDNECENGGFCSGVCHNLPGTFECICGPDSALVRHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 2 - protein, 515 aa, HOMO SAPIENS
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 3 - protein, 484 aa, HOMO SAPIENS
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGK

SEQ ID NO: 4 - protein, 497 aa, HOMO SAPIENS
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 5 - protein, 466 aa, HOMO SAPIENS
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGK

SEQ ID NO: 6 - protein, 466 aa, HOMO SAPIENS
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDADECENGGFCSGVCHNLPGTFECICGPDSALARHIGTDCDSGK

SEQ ID NO: 7 - DNA, 1398 bp, HOMO SAPIENS
gcccctgccgagcctcagcctggcggcagccagtgcgtggagcacgactgcttcgccctgtaccccggacccgccaccttcctgaacgccagccagatctgcgacggcctgcggggccacctgatgaccgtgcggagcagcgtggccgccgacgtgatcagcctgctgctgaacggcgacggcggcgtgggcaggcggaggctgtggatcggactgcagctgccccctggctgcggcgaccccaagaggctgggccccctgcggggcttccagtgggtgaccggcgacaacaacaccagctacagcagatgggccaggctggacctgaatggcgcccctctgtgcggcccactgtgcgtggccgtgtctgccgccgaggccaccgtgcccagcgagcccatctgggaggaacagcagtgcgaagtgaaggccgacggcttcctgtgcgagttccacttccccgccacctgcaggcctctggccgtggaacctggagccgctgctgccgccgtgagcatcacctacggcacccccttcgccgccagaggcgccgacttccaggccctgcccgtgggctcttctgccgccgtggcccccctggggctgcagctgatgtgcaccgcccctccaggcgccgtgcagggccactgggccagagaagcccctggcgcctgggactgcagcgtggagaacggcggctgcgagcacgcctgcaacgccatccctggcgcccctaggtgccagtgccctgccggagccgccctccaggccgatggcagaagctgcaccgccagcgccacccagagctgcaacgacctgtgcgagcacttctgcgtgcccaaccccgaccagcccggcagctacagctgcatgtgcgagaccggctaccggctggccgccgatcagcacagatgcgaggacgtggacgactgcatcctggaacccagcccctgcccccagagatgcgtgaacacccagggcggcttcgagtgccactgctaccccaactacgacctggtggacggcgagtgtgtggagcccgtggacccctgcttccgggccaactgcgagtaccagtgccagcccctgaaccagaccagctacctgtgcgtgtgcgccgaaggcttcgcccccatcccccacgagccccaccggtgccagatgttctgcaaccagaccgcctgccctgccgactgcgaccccaatacccaggccagctgcgagtgccccgagggctacatcctggacgacggcttcatctgcaccgacatcgacgagtgcgagaatggcggcttctgcagcggcgtgtgccacaacctgcccggcaccttcgagtgcatctgcggccctgacagcgccctggcccggcacatcggcaccgactgcgatagcggcaag

SEQ ID NO: 8 - DNA, 1398 bp, HOMO SAPIENS
gcccctgccgagcctcagcctggcggcagccagtgcgtggagcacgactgcttcgccctgtaccccggacccgccaccttcctgaacgccagccagatctgcgacggcctgcggggccacctgatgaccgtgcggagcagcgtggccgccgacgtgatcagcctgctgctgaacggcgacggcggcgtgggcaggcggaggctgtggatcggactgcagctgccccctggctgcggcgaccccaagaggctgggccccctgcggggcttccagtgggtgaccggcgacaacaacaccagctacagcagatgggccaggctggacctgaatggcgcccctctgtgcggcccactgtgcgtggccgtgtctgccgccgaggccaccgtgcccagcgagcccatctgggaggaacagcagtgcgaagtgaaggccgacggcttcctgtgcgagttccacttccccgccacctgcaggcctctggccgtggaacctggagccgctgctgccgccgtgagcatcacctacggcacccccttcgccgccagaggcgccgacttccaggccctgcccgtgggctcttctgccgccgtggcccccctggggctgcagctgatgtgcaccgcccctccaggcgccgtgcagggccactgggccagagaagcccctggcgcctgggactgcagcgtggagaacggcggctgcgagcacgcctgcaacgccatccctggcgcccctaggtgccagtgccctgccggagccgccctccaggccgatggcagaagctgcaccgccagcgccacccagagctgcaacgacctgtgcgagcacttctgcgtgcccaaccccgaccagcccggcagctacagctgcatgtgcgagaccggctaccggctggccgccgatcagcacagatgcgaggacgtggacgactgcatcctggaacccagcccctgcccccagagatgcgtgaacacccagggcggcttcgagtgccactgctaccccaactacgacctggtggacggcgagtgtgtggagcccgtggacccctgcttccgggccaactgcgagtaccagtgccagcccctgaaccagaccagctacctgtgcgtgtgcgccgaaggcttcgcccccatcccccacgagccccaccggtgccagatgttctgcaaccagaccgcctgccctgccgactgcgaccccaatacccaggccagctgcgagtgccccgagggctacatcctggacgacggcttcatctgcaccgacgccgacgagtgcgagaatggcggcttctgcagcggcgtgtgccacaacctgcccggcaccttcgagtgcatctgcggccctgacagcgccctggcccggcacatcggcaccgactgcgatagcggcaag

SEQ ID NO: 9 - DNA, 1545 bp, HOMO SAPIENS
atgctgggcgtgctggtgctgggagccctggccctggccggcctgggctttcctgcccctgccgagcctcagcctggcggcagccagtgcgtggagcacgactgcttcgccctgtaccccggacccgccaccttcctgaacgccagccagatctgcgacggcctgcggggccacctgatgaccgtgcggagcagcgtggccgccgacgtgatcagcctgctgctgaacggcgacggcggcgtgggcaggcggaggctgtggatcggactgcagctgccccctggctgcggcgaccccaagaggctgggccccctgcggggcttccagtgggtgaccggcgacaacaacaccagctacagcagatgggccaggctggacctgaatggcgcccctctgtgcggcccactgtgcgtggccgtgtctgccgccgaggccaccgtgcccagcgagcccatctgggaggaacagcagtgcgaagtgaaggccgacggcttcctgtgcgagttccacttccccgccacctgcaggcctctggccgtggaacctggagccgctgctgccgccgtgagcatcacctacggcacccccttcgccgccagaggcgccgacttccaggccctgcccgtgggctcttctgccgccgtggcccccctggggctgcagctgatgtgcaccgcccctccaggcgccgtgcagggccactgggccagagaagcccctggcgcctgggactgcagcgtggagaacggcggctgcgagcacgcctgcaacgccatccctggcgcccctaggtgccagtgccctgccggagccgccctccaggccgatggcagaagctgcaccgccagcgccacccagagctgcaacgacctgtgcgagcacttctgcgtgcccaaccccgaccagcccggcagctacagctgcatgtgcgagaccggctaccggctggccgccgatcagcacagatgcgaggacgtggacgactgcatcctggaacccagcccctgcccccagagatgcgtgaacacccagggcggcttcgagtgccactgctaccccaactacgacctggtggacggcgagtgtgtggagcccgtggacccctgcttccgggccaactgcgagtaccagtgccagcccctgaaccagaccagctacctgtgcgtgtgcgccgaaggcttcgcccccatcccccacgagccccaccggtgccagatgttctgcaaccagaccgcctgccctgccgactgcgaccccaatacccaggccagctgcgagtgccccgagggctacatcctggacgacggcttcatctgcaccgacatcgacgagtgcgagaatggcggcttctgcagcggcgtgtgccacaacctgcccggcaccttcgagtgcatctgcggccctgacagcgccctggcccggcacatcggcaccgactgcgatagcggcaaggtggacgggggcgactccggctccggcgagccccctcccagccctacccccggcagcaccctgacccctcccgccgtgggcctggtgcacagc

SEQ ID NO: 10 - DNA, 1545 bp, HOMO SAPIENS
atgctgggcgtgctggtgctgggagccctggccctcgctggactgggctttcctgcccctgccgagcctcagcctggcggcagccagtgcgtggagcacgactgcttcgccctgtaccccggacccgccaccttcctgaacgccagccagatctgcgacggcctgagaggccacctgatgaccgtgcggagcagcgtggccgccgacgtgatcagcctgctgctgaacggcgacggcggcgtgggcaggcggagactgtggatcggcctgcagctgccccctggctgcggcgaccccaagagactgggccccctgcggggcttccagtgggtgaccggcgacaacaacaccagctacagcagatgggccagactggacctgaatggcgcccctctgtgcggcccactgtgcgtggccgtgtctgctgccgaggccaccgtgcccagcgagcccatctgggaggaacagcagtgcgaagtgaaggccgacggcttcctgtgcgagttccacttccccgccacctgcagacccctggccgtggaacccggcgccgctgctgcagccgtgtctatcacctacggcacccccttcgccgccagaggcgccgacttccaggccctgcccgtgggaagctctgccgccgtggcccctctggggctgcagctgatgtgcaccgcccctccaggcgccgtgcagggccactgggccagagaagcccctggggcctgggactgcagcgtggagaacggcggctgcgagcacgcctgcaacgccatccctggcgcccctagatgccagtgccctgctggagccgccctgcaggccgatggcagaagctgcaccgccagcgccacccagagctgcaacgacctgtgcgagcacttctgcgtgcccaaccccgaccagcctggaagctacagctgcatgtgcgagacaggctaccggctggccgccgatcagcacagatgcgaggacgtggacgactgcatcctggaacccagcccctgcccccagagatgcgtgaacacccagggcggcttcgagtgccactgctaccctaactacgacctggtggacggcgagtgtgtggagcccgtggacccctgcttccgggccaactgcgagtaccagtgccagcccctgaaccagaccagctacctgtgcgtgtgcgccgaaggcttcgcccccatcccccacgagccccaccggtgccagatgttctgcaaccagaccgcctgtcctgccgactgcgaccccaatacccaggccagctgtgagtgccccgagggctacatcctggacgacggcttcatctgcacagacgccgacgagtgcgagaatggcggcttctgcagcggcgtgtgccacaacctgcccggcaccttcgagtgcatctgcggccctgacagcgccctggccagacacatcggcaccgactgcgatagcggcaaggtggacgggggggacgccggagccggcgagcctccccccagccctacccccggcagcaccctgacccctcccgccgtgggcctggtgcacagc

SEQ ID NO: 11 - protein, 426 aa, HOMO SAPIENS
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDE

SEQ ID NO: 12 - DNA, 1278 bp, HOMO SAPIENS
gcccctgccgagcctcagcctggcggcagccagtgcgtggagcacgactgcttcgccctgtaccccggacccgccaccttcctgaacgccagccagatctgcgacggcctgcggggccacctgatgaccgtgcggagcagcgtggccgccgacgtgatcagcctgctgctgaacggcgacggcggcgtgggcaggcggaggctgtggatcggactgcagctgccccctggctgcggcgaccccaagaggctgggccccctgcggggcttccagtgggtgaccggcgacaacaacaccagctacagcagatgggccaggctggacctgaatggcgcccctctgtgcggcccactgtgcgtggccgtgtctgccgccgaggccaccgtgcccagcgagcccatctgggaggaacagcagtgcgaagtgaaggccgacggcttcctgtgcgagttccacttccccgccacctgcaggcctctggccgtggaacctggagccgctgctgccgccgtgagcatcacctacggcacccccttcgccgccagaggcgccgacttccaggccctgcccgtgggctcttctgccgccgtggcccccctggggctgcagctgatgtgcaccgcccctccaggcgccgtgcagggccactgggccagagaagcccctggcgcctgggactgcagcgtggagaacggcggctgcgagcacgcctgcaacgccatccctggcgcccctaggtgccagtgccctgccggagccgccctccaggccgatggcagaagctgcaccgccagcgccacccagagctgcaacgacctgtgcgagcacttctgcgtgcccaaccccgaccagcccggcagctacagctgcatgtgcgagaccggctaccggctggccgccgatcagcacagatgcgaggacgtggacgactgcatcctggaacccagcccctgcccccagagatgcgtgaacacccagggcggcttcgagtgccactgctaccccaactacgacctggtggacggcgagtgtgtggagcccgtggacccctgcttccgggccaactgcgagtaccagtgccagcccctgaaccagaccagctacctgtgcgtgtgcgccgaaggcttcgcccccatcccccacgagccccaccggtgccagatgttctgcaaccagaccgcctgccctgccgactgcgaccccaatacccaggccagctgcgagtgccccgagggctacatcctggacgacggcttcatctgcaccgacatcgacgag

SEQ ID NO: 13 - protein, 18 aa, Homo sapiens
MLGVLVLGALALAGLGFP

(19) United States
(12) Patent Application Publication
(10) Pub. No.: US 2011/0207670 A1
(43) Pub. Date: Aug. 25, 2011

(54) COMPOSITIONS AND METHODS OF USE FOR SOLUBLE THROMBOMODULIN VARIANTS

(75) Inventors: David Thompson Berg, Beech Grove, IN (US); Brian William Grinnell, Indianapolis, IN (US); Akanksha Gupta, Indianapolis, IN (US)

(73) Assignee: ELI LILLY AND COMPANY, Indianapolis, IN (US)

(21) Appl. No.: 13/126,474

(22) PCT Filed: Oct. 21, 2009
(86) PCT No.: PCT/US09/61407

§ 371 Date: Apr. 28, 2011

Related U.S. Application Data
(60) Provisional application No. 61/113,801, filed on Nov. 12, 2008.

(51) Int. Cl. A61K 38/17; C07K 14/435; A61P 13/12

(52) U.S. Cl. 514/15.4; 530/350

(57) ABSTRACT

The present invention provides a method for preventing and/or treating a patient with acute kidney injury caused by a variety of conditions. The method comprises administering to the patient soluble thrombomodulin variants that do not bind thrombin. In conjunction with standard of care, soluble thrombomodulin variants that do not bind thrombin will prevent or reduce acute kidney injury and subsequent morbidity and mortality.

This invention relates to medical science, particularly the treatment of microvascular dysfunction with variants of soluble thrombomodulin. More specifically, the present invention concerns the treatment of microvascular dysfunction as occurs in acute kidney injury by administering to a patient in need thereof a variant of soluble thrombomodulin that does not bind thrombin.

Thrombomodulin (TM) is a glycoprotein anchored on the membrane surface of endothelial cells on many organs, including lung, liver, and kidney and plays an important role in vascular response to injury. TM binds to thrombin and modulates both coagulation and inflammatory activation.

TM is composed of five domains: an N-terminal lectin-like binding domain, an epidermal growth factor (EGF) domain which consists of 6 EGF-like repeats, a Ser/Thr-rich region, a transmembrane domain and a cytoplasmic domain. Soluble TM (sTM) variants have been constructed by deleting the cytoplasmic and transmembrane domains. TM deletion variants have been used to determine the smallest TM fragment (EGF-like repeats 4-6) which retains thrombin binding and subsequent cleavage of protein C by the thrombin-TM complex. Alanine scanning of this TM region was done to determine residues which are critical for thrombomodulin activity (WO 93/25675). Changes of specific residues to alanine within polypeptides consisting of EGF 4-6 resulted in sTM variants with a modified cofactor activity upon binding to thrombin. Proposed therapeutic applications included the inhibition of clot formation and treatment of systemic coagulation disorders such as disseminated intravascular coagulation. However, such applications carry the inherent risk of bleeding complications due to the disruption of the coagulation cascade. More recently, Ikeguchi et al. (Kidney International, 2002, 61:490-501) reported on the effects of sTM on experimental glomerulonephritis and concluded that the anti-thrombotic action of sTM effectively attenuated the injuries of thrombotic glomerulonephritis.

Acute kidney injury (AKI) is a general term referring to conditions resulting from an acute insult to the microvasculature of the kidney. This microvascular dysfunction may be associated with infection/inflammation, ischemic injury, contrast agents, or chemotherapeutics. AKI is generally characterized by a sudden decline in glomerular filtration rate, the accumulation of nitrogen wastes and the inability of the kidney to regulate electrolyte and water balance.

Despite the technical advances in the care of patients who suffer from AKI and improvements in the understanding of the pathophysiology of the disease process, there is still a high morbidity and mortality associated with this condition. Recent trials of therapeutic agents for AKI have not been successful. Thus, an unmet medical need exists for the treatment of AKI that is both safe and effective.

Unexpectedly, applicants have discovered that soluble thrombomodulin variants that do not bind thrombin, are particularly effective in protecting the kidney from injury in in vivo experimental models of AKI. A soluble thrombomodulin variant that does not bind thrombin offers a potentially significant alternative approach to the prevention and treatment of AKI without the potential bleeding complications that can result from modulation of the coagulation cascade.

The present invention provides a method of treating AKI in a patient in need thereof which comprises administering to the patient an effective amount of an sTM variant that does not bind thrombin wherein the variant has a Kd value of >4300 for thrombin binding under the BIAcore assay conditions described in Example 3. Preferred sTM variants of this method comprise the amino acid sequences shown in SEQ ID NO: 6 or SEQ ID NO: 11.

The present invention also provides a method of preventing AKI in a patient susceptible to AKI comprising administering to the patient an effective amount of an sTM variant that does not bind thrombin wherein the variant has a Kd value of >4300 for thrombin binding under the BIAcore assay conditions described in Example 3. Preferred sTM variants of this method comprise the amino acid sequences shown in SEQ ID NO: 6 or SEQ ID NO: 11.

The present invention provides an sTM variant that does not bind thrombin for use in treating AKI.

The present invention also provides sTM variants that do not bind thrombin for use in treating AKI wherein the variant has a Kd value of >4300 for thrombin binding under the BIAcore assay conditions described in Example 3. Preferred sTM variants of this use comprise the amino acid sequences shown in SEQ ID NO: 6 or SEQ ID NO: 11.

The present invention further provides the use of an sTM variant that does not bind thrombin for preventing AKI.

The present invention also provides an sTM variant that does not bind thrombin for use in preventing AKI wherein the variant has a Kd value of >4300 for thrombin binding under the BIAcore assay conditions described in Example 3. Preferred sTM variants of this use comprise the amino acid sequences shown in SEQ ID NO: 6 or SEQ ID NO: 11.

The present invention also provides an sTM variant comprising the amino acid sequence shown in SEQ ID NO: 11.

The present invention further provides an sTM variant that does not bind thrombin for use as a medicament wherein said variant comprises the amino acid sequence shown in SEQ ID NO: 11.

The present invention also provides an sTM variant that does not bind thrombin for use as a medicament said variant is as shown n SEQ ID NO: 11.

The present invention provides sTM variants that do not bind thrombin for use in the manufacture of a medicament for treating AKI. The present invention further provides the use of an sTM variant, which does not bind thrombin for the manufacture of a medicament for preventing AKI. Preferred sTM variants of this use comprise the amino acid sequences shown in SEQ ID NO: 6 or SEQ ID NO: 11.

The present invention also provides for a pharmaceutical composition comprising an sTM variant having the amino acid sequence shown in SEQ ID NO: 11 and a pharmaceutically acceptable excipient.

The present invention provides isolated polynucleotides that encode the amino acid sequences shown in SEQ ID NO: 6 or SEQ ID NO: 11, wherein said polynucleotides comprise the sequences of SEQ ID NO:8 or SEQ ID NO: 12. The invention further provides a recombinant expression vector comprising said isolated polynucleotide and a host cell transfected with the recombinant expression vector.

The present invention provides a process for producing an sTM variant having the amino acid sequence shown in SEQ ID NO: 11 comprising cultivating the host cell stably transfected with the recombinant expression vector, expressing said polypeptide, and recovering the polypeptide encoded by said polynucleotide from the culture.

For purposes of the present invention, as disclosed and claimed herein, the following terms are as defined below.

“AKI” refers to acute kidney injury which has as one symptom an acute reduction in glomerular filtration rate associated with the retention of nitrogenous wastes. The reduction may be due to an AKI such as acute tubular necrosis or acute interstitial nephritis. AKI alternatively may be referred to as acute renal dysfunction.

“Effective amount” refers to an amount necessary (at dosages and for periods of time and for the means of administration) to achieve the desired therapeutic result. An effective amount of the sTM variant may vary according to factors such as the disease state, age, sex, and weight of the individual, and the ability of the sTM variant to elicit a desired response in the individual.

“Treating” or “treat” means the management and care of a patient to eliminate, reduce, or control a disease, condition, or disorder.

“Preventing” or “prevent” describes the management and care of a patient to delay onset of the symptoms or complications of a disease, condition, or disorder.

“Patient” means a mammal, preferably a human, that has or that is susceptible to having or developing AKI. In certain indications, the patient has microvascular dysfunction as occurs with AKI that would benefit from treatment with an sTM variant that does not bind thrombin.

Soluble thrombomodulin (sTM) refers to soluble thrombomodulin of SEQ ID NO:4 or SEQ ID NO:5. sTM is a soluble, secreted variant of thrombomodulin which lacks the full-length thrombomodulin transmembrane and cytoplasmic domains The primary amino acid structure of human thrombomodulin (SEQ ID NO: 1) is known in the art, as described in EP 0412841. Human TM is synthesized as a 575 amino acid protein including a signal peptide portion reported to be 16, 18, or 21 residues in length. Following the signal peptide portion, human TM comprises the following domains or regions, sequentially from the amino terminus: 1) an amino terminal domain of ˜222-226 amino acids, 2) six EGF (“epidermal growth factor”)-like structures totaling ˜236-240 amino acids (EGF domain), 3) a serine/threonine rich domain (ST domain) of ˜34-37 amino acids, having several possible O-glycosylation sites, 4) a transmembrane region of ˜23-24 amino acids, and 5) a cytoplasmic domain of ˜36-38 amino acids. As used herein, “amino terminal domain,” “EGF domain,” “ST domain,” “transmembrane region' or domain,” and “cytoplasmic region' or domain” refer to the approximate range of amino acid residues noted above for each region or domain. Further, because in vivo processing will be expected to vary depending upon the expressing transformed host cell, especially a prokaryotic host cell compared to a eukaryotic host cell, the term “amino terminal region or domain” optionally may include the thrombomodulin signal peptide or portion thereof. For example, spTMD1 (SEQ ID NO:2) contains the signal peptide, amino terminal domain, EGF domain, and the ST domain, while spTMD2 (SEQ ID NO:3) contains the signal peptide, amino terminal domain, and the EGF domain.

“sTM variant” refers to sTM with one or more substitutions in the EGF5 domain or deletion of the EGF6 domain when compared to SEQ ID NO: 4 or SEQ ID NO:5. sTM variants can be generated by procedures known in the art and assayed for their lack of ability to bind thrombin by standard procedures such as the BIACore assay described in Example 3. Under these assay conditions, the Kd value of >4300 is beyond the detection limit of the assay and therefore indicate that the sTM variants do not bind to thrombin.

Microvascular dysfunction is a general term that refers to the dysfunction of the microcirculation in any organ i.e. kidney, heart, lung, etc., that may occur affecting both blood pressure and flow patterns that may have consequences for peripheral vascular resistance. Microcirculation includes the smallest arteries and arterioles in addition to capillaries and venules.

Amino acid position numbering is based on SEQ ID NO: 4, also designated as TMD1, which contains the amino terminal domain, EGF domain, and the ST domain but lacks the signal peptide. The first alanine of SEQ ID NO:4 is designated position 1 for numbering of amino acids. The wild-type full-length soluble human thrombomodulin that is truncated immediately after EGF6 and lacks the signal peptide is SEQ ID NO:5, also designated asTMD2.

Further, the sTM variants of the present invention are named as follows: one letter code for the substituted amino acid, the amino acid position number, followed by the replacing amino acid residue. For example, I424A, refers to an sTM variant in which the isoleucine at position 424 has been changed to an alanine.

The sTM variants of the present invention do not bind thrombin. Preferably, the sTM variant that does not bind thrombin is TMD2 (SEQ ID NO:5) in which the isoleucine at position 424 is substituted with alanine. This variant may also be referred to asTMD2-I424A (SEQ ID NO:6).

In another embodiment, the sTM variant that does not bind thrombin is a truncated form of TMD2 in which the EGF 6 domain is removed and is designated as TM-LE15 (SEQ ID NO: 11).

Methods to produce human recombinant sTM and variants of human TM have been described previously (Parkinson et al., 1990 J. Biol. Chem. 265:12602-12610 and Nagashima et al., 1993 J. Biol. Chem. 268: 2888-2892). sTM variants utilized in this invention are the result of molecular genetic manipulations that can be achieved in a variety of ways known in the art. DNA sequences are derived from the amino acid sequences of the sTM variants of the present invention by procedures well known in the art. Preferred DNA sequences of the present invention are the DNA sequences encoding TMD2 (SEQ ID NO: 7), TMD2-I424A (SEQ ID NO:8), and TM-LE15 (SEQ ID NO: 12). Additionally, the DNA sequences encoding the sTM variants of the present invention are incorporated into plasmids or expression vectors which in turn are transfected into recombinant cells to provide a means to produce pharmaceutically useful compounds wherein the compound, secreted from recombinant cells, is an sTM variant. It is understood that the DNA sequences of the present invention may also encode a leader sequence such as the leader sequence of SEQ ID NO:13.

The sTM variants of the present invention may readily be produced in mammalian cells such as CHO, NS0, HEK293 or COS cells, in bacterial cells such as E. coli, or in fungal or yeast cells. The host cells are transfected with an expression vector that contains an sTM variant and are then cultured using techniques well known in the art.

The protein expressed from the host cells is recovered from the culture media and purified. Various methods of protein purification may be employed and such methods are known in the art and described, for example, in Deutscher, Methods in Enzymology 182: 83-89 (1990) and Scopes, Protein Purification: Principles and Practice, 3^{rd} Edition, Springer, N.Y. (1994).

Generally, the definitions of nomenclature and descriptions of general laboratory procedures described in this application can be found in J. Sambrook et al., Molecular Cloning, A Laboratory Manual, (1989) Cold Spring Harbor Laboratory, Cold Spring Harbor, N.Y. The manual is hereinafter referred to as Sambrook. In addition, Ausubel et al., eds., Current Protocols in Molecular Biology, (1987 and periodic updates) Greene Publishing Associates, Wiley-Interscience, New York, discloses methods useful in the present application.

sTM variants are generated by altering or truncating the amino acid sequence of human sTM SEQ ID NO:4 or SEQ ID NO:5. Methods by which amino acids can be removed or replaced in the sequence of a protein are well known. See, e.g., Sambrook, supra; Ausubel et al., supra and references cited therein.

An assay measuring protein C activation by a thrombin/thrombomodulin complex is used to determine thrombin binding by the sTM variants of the present invention. During coagulation human protein C is activated by thrombin. However, this activation reaction is slow unless thrombin is complexed with thrombomodulin. The assay depicted in Example 1 shows that the human thrombin and sTM complex activates human protein C. However, human protein C activation is not detectable in the presence of human thrombin and the sTM variants TMD2-I424A or TM-LE15, indicating that TMD2-I424A and TM-LE15 do not bind thrombin and therefore fail to activate protein C.

Additional methods demonstrating that the variants of sTM of the present invention do not bind thrombin are an assay showing thrombomodulin inhibition of thrombin induced C++ flux in human umbilical vein endothelial cells (Example 2) and BIAcore analysis of the binding kinetics and affinity of the sTM/thrombin complex (Example 3).

In vivo models that are indicative of the effectiveness of sTM variants of the present invention to reduce or prevent AKI are shown in Examples 4 and 5.

For example, a LPS-induced AKI rat model that was run essentially as described by Kikeri et al., (1986 Am. J. Physiol. 250:F1098-F1106) is represented in Example 4. The LPS-induced model generally consists of inducing AKI with a bolus injection of E. coli LPS. The bolus injection of LPS causes endotoxemia, resulting in a decrease in glomerular rate function and an increase in blood-urea-nitrogen (BUN) levels. sTM variants are tested for their ability to reduce or prevent this AKI by treating the rats with human sTM variants prior to induction of endotoxemia. As shown in Table 4, administration of human sTM or a variant of human sTM that does not bind thrombin, are able to reduce AKI as measured by reduction in BUN levels.

In addition, a rat bilateral renal artery clamp model is done as described in Example 5. In this model, bilateral renal ischemia is induced by clamping the renal pedicles, with reperfusion injury resulting when the clamps are removed. A measurement of renal damage is an increase in serum creatinine levels. As shown in Table 5, administration of variants of human sTM that do not bind thrombin, reduce AKI as indicated by a reduction in serum creatinine levels.

Pharmaceutical compositions of the present invention may be administered by any means known in the art that achieve the generally intended purpose to treat AKI. The preferred route of administration is parenteral, defined herein as referring to modes of administration that include intravenous, intramuscular, intraperitoneal, intrasternal, subcutaneous, and intraarticular injection and infusion. More preferably, sTM variants will be administered either by IV bolus and/or subcutaneous injection. Preferred exposure times range from one to 24 or more hours, including but not limited to 48, 72, 96, or as many as 120 hours. The dosage administered will be dependent upon the age, health, and weight of the recipient, kind of concurrent treatment, if any, frequency of treatment, and the nature of the effect desired. Typical dosage levels can be optimized using standard clinical techniques and will be dependent on the mode of administration and the condition of the patient. Generally, doses will be in the range of 1 μg/kg to 10 mg/kg; 2.5 μg/kg to 5 mg/kg; 5 μg/kg to 2.5 mg/kg; or 10 μg/kg to 500 μg/kg.

The pharmaceutical compositions must be appropriate for the selected mode of administration, and pharmaceutically acceptable excipients such as, buffers, surfactants, preservatives, solubilizing agents, isotonicity agents, stabilizing agents, carriers, and the like are used as appropriate. Remington's Pharmaceutical Sciences, Mack Publishing Co., Easton Pa.

The concentration of the sTM variant in formulations may be from as low as about 0.1% (1 mg/mL) to as much as 15% or 20% (150 to 200 mg/mL) and will be selected primarily based on fluid volumes, viscosities, stability, and so forth, in accordance with the particular mode of administration selected. Preferred concentrations of the sTM variant will generally be in the range of 1 to about 100 mg/mL.

The following examples are intended to illustrate but not to limit the invention.

### EXAMPLE 1

### Human Protein C Activation by sTM Variants

A kinetic analysis is done to determine the rate of activation of human protein C by human sTM variants. For each sTM variant, the reactions are set up as follows. In a final 100 μL reaction volume, human protein C is added to a final concentration of 150 nM and human thrombin is added to a final concentration of 2 nM. sTM controls TMD1 and TMD2 or an sTM variant in AB/BSA buffer (150 mM NaCl; 20 mM Tris pH 7.5; 3 mM CaCl₂; 1 mg/mL BSA) is added to the reaction at final concentrations varying from 0 nM to 250 nM. The reaction mixture is incubated 30 minutes at 37° C. 25 μL of each reaction is removed to a 96 well plate containing 150 μL of Thrombin Stop Buffer (1 unit/ml hirudin in 150 mM NaCl; 20 mM Tris pH 7.5; 3 mM CaCl₂) and incubated for 5 minutes at room temperature. 25 μL of a 4 μM stock solution of S2366 chromogenic substrate (L-Pyroglutamyl-L-prolyl-L-arginine-p-Nitroaniline hydrochloride, Chromogenix) is added and mixed briefly. The Optical Density at 405 nm (OD405) is read on a microplate spectrophotometer in a five minute kinetic read with a 6 second read interval. Michaelis Menton kinetic constants are calculated from the kinetic data using SigmaPlot software with the Enzyme Kinetics Module 1.1. This method was based in part on the protocol described in Grinnell et al., 1994 Biochem J. 303: 929-933.

As shown in Table 1, human TMD1 (SEQ ID NO: 4) and human TMD2 (SEQ ID NO: 5) are able to activate human protein C and a Kd for binding to thrombin is determined. This dissociation constant, Kd, indicates the strength of binding between human protein C and thrombin in terms of how easy it is to separate the complex (dissociation or ‘off rate’). The rate of human protein C activation obtained with the sTM variants I424A (SEQ ID NO: 6) or TM-LE15 (SEQ ID NO: 11) and thrombin was very low, and no Kd for thrombin binding could be calculated (TLD=Too Low for Detection). This indicates that the sTM variants I424A (SEQ ID NO: 6) and TM-LE15 (SEQ ID NO: 11) are unable to bind to thrombin and activate human protein C.

TABLE 1

| sTM variant | Vmax (mOD405/min) | Kd |
|---|---|---|
| TMD1 | 43.3 | 6.9 |
| (SEQ ID NO: 4) | | |
| TMD2 | 39.1 | 16.2 |
| (SEQ ID NO: 5) | | |
| TMD2-I424A | 6.9 | TLD |
| (SEQ ID NO: 6) | | |
| TM-LE15 | 8.3 | TLD |
| (SEQ ID NO: 11) | | |

### EXAMPLE 2

### Thrombomodulin Inhibition of Thrombin Induced Ca⁺⁺Flux in HUVEC

A compound such as sTM that binds to thrombin interferes with the binding of thrombin to HUVEC cells and the subsequent induction of Ca++. An in vitro assay is used to evaluate the effect of sTM variants on thrombin induced Ca++flux in HUVEC. A black-well, clear-bottom 96-well plate is seeded with 10⁴ human umbilical vein endothelial cells and incubated for 48 hours at 37° C., 5% CO₂. After the two day incubation, 100 μL/well of Loading Buffer from a FLIPR Calcium 4 Assay Kit (Molecular Devices, Cat. # R8142) is added following the manufacture's protocol. A reagent containing 5 nM human thrombin with or without 500 nM soluble thrombomodulin (TMD1, TMD2 or TMD2-I424A) in Hank's Buffered Saline Solution, 20 nM HEPES and 0.75% Bovine Albumin Fraction V is then added at 100 μL/well and incubated for 30 minutes at room temperature. Thrombin induced Ca++flux was measured on a Fluorescent Imaging Plate Reader-2 (Molecular Devices, Sunnyvale, Calif.). It is evident from Table 2 that the sTM variant TMD2-I424A does not bind thrombin when compared to sTM variants TMD1 and TMD2 as measured by the induction of Ca++influx in HUVEC.

TABLE 2

| Compound | Area Under Curve | St. Dev. |
|---|---|---|
| No sTM | 5408 | 780 |
| TMD1 | 2533 | 316 |
| (SEQ ID NO: 4) | | |
| TMD2 | 2639 | 257 |
| (SEQ ID NO: 5) | | |
| TMD2-I424A | 5842 | 355 |
| (SEQ ID NO: 6) | | |

### EXAMPLE 3

### Evaluation of sTM Variants Binding to Thrombin by Surface Plasmon Reasonance (BIAcore)

The thrombin binding properties of various human sTM variants are determined using a BIAcore biosensor 2000 instrument. All measurements are performed at room temperature. All experiments are performed in HBS-P (10 mM HEPES, pH 7.4, containing 150 mM NaCl) buffer containing 5 mM CaCl₂. For all binding experiments, biotinylated sTM variants are immobilized on an SA (streptavidin-coated) sensor chip at a level of 200 to 300 response units (RU). Biotinylated sTM variants are prepared by incubating 10 μM sTM variant (0.5 mL) with 50 μM NHS-LC-Biotin for 2 hours at room temperature, followed by dialysis against phosphate buffered saline overnight. The binding of human thrombin (PPACK-inhibited, Enzyme Research Laboratories), mouse thrombin, and rat thrombin are tested.

The binding of thrombin is evaluated using multiple analytical cycles. Each cycle is performed at a flow rate of 100 μL/minute and consists of the following steps: injection of 250 μL of a solution of varying thrombin concentration with two injections for each concentration followed by a 1 minute delay for dissociation. Following the dissociation phase, the biosensor chip surface was regenerated using an injection of 50 μL of 5 mM EDTA, followed by similar injection of running buffer. Concentrations of thrombin ranged from 200 nM to 1.6 nM, and were prepared by serial, two-fold dilutions, starting with 200 nM thrombin. Due to the rapid association and dissociation rates, the equilibrium binding affinities are determined using the steady-state signals, which are obtained by averaging the signal over the final 30 sec of the injection phase; the resulting signal versus thrombin concentration data were then fit to a 1 to 1 equilibrium binding model using Scrubber (Center for Biomolecular Interaction Analysis, Univ. of Utah). All traces are referenced to a control surface in which 10 μM biotin was injected over the streptavidin surface. It is evident from Table 3 that sTM variants TMD2-I424A (SEQ ID NO: 6) and TM-LE15 (SEQ ID NO: 11) do not bind thrombin when compared to sTM variants TMD1 and TMD2. Under the assay conditions described above, the Kd value of >4300 is beyond the detection limit of the assay and therefore indicate that the sTM variants of SEQ ID NO: 6 and SEQ ID NO: 11 do not bind to thrombin.

TABLE 3

| | Human IIa |
|---|---|
| sTM Variant | K_{D} (nM) |
| TMD1 | 10.3 ± 0.1 |
| (SEQ ID NO: 4) | |
| TMD2 | 10.1 ± 0.3 |
| (SEQ ID NO: 5) | |
| TMD2-I424A | >4300 |
| (SEQ ID NO: 6) | |
| TM-LE15 | >4300 |
| (SEQ ID NO: 11) | |

### EXAMPLE 4

### Efficacy of Human and Rat sTM in LPS-Induced Acute Renal Failure in Rats

An LPS-induced AKI rat model is run essentially as described by Kikeri et al., (1986 Am. J. Physiol. 250:F1098-F1106). The LPS-induced model generally consists of inducing AKI with a bolus injection of E. coli LPS. The bolus injection of LPS causes endotoxemia, resulting in a decrease in glomerular rate function and an increase in blood-urea-nitrogen (BUN) levels. sTM variants may be tested for their ability to reduce or prevent this AKI by treating the rats with human sTM variants prior to induction of endotoxemia.

Male Sprague-Dawley rats (Harlan, Ind., USA) weighing 200-250 g are used in the study. The animals are randomized into two groups at the time of surgery: 1) saline treated control and 2) LPS-treated animals. The animals in the LPS-treated group are further divided into subgroups vehicle, TMD2 (SEQ ID NO: 5), or human sTM variant TMD2-I424A (SEQ ID NO: 6). Endotoxemia is induced by intraperitoneal administration of E. coli LPS (20 mg/kg). The control group receives pyrogen-free saline. TMD2 (5 mg/kg and 2.5 mg/kg) or TMD2-I424A (5 mg/kg and 2.5 mg/kg) are administered subcutaneously 12-h prior to the induction of endotoxemia. Animals are sacrificed 24-h post-LPS administration and blood samples are collected for BUN analysis. The results below are the average of 4 rats per group.

As shown in Table 4, administration of human sTM or a variant of human sTM that does not bind thrombin, are able to reduce AKI as measured by reduction in BUN levels. sTM variant TMD2-I424A (SEQ ID NO: 6) reduces BUN levels more effectively than TMD2 at comparable levels.

TABLE 4

| Group | BUN (mg/dl) |
|---|---|
| Control | 10 |
| LPS | 73 |
| LPS + | 25 |
| TMD2 | |
| 5 mg/kg | |
| LPS + | 12 |
| TMD2-I424A | |
| 5.0 mg/kg | |
| LPS + | 45 |
| TMD2 | |
| 2.5 mg/kg | |
| LPS + | 25 |
| TMD2-I424A | |
| 2.5 mg/kg | |

### EXAMPLE 5

### Bilateral Renal Artery Clamp Model for AKI

Male Sprague-Dawley rats weighing 180-250 g are anesthetized utilizing 5% isoflurane for induction and 1.5% for maintenance and placed on a homeothermic table to maintain core body temperature at 37° C. A jugular vein is cannulated with PE50 catheter for infusion of TMD2-I424A (SEQ ID NO: 6) or TM-LE15 (SEQ ID NO: 11). A midline incision is made, the renal pedicles are isolated, and bilateral renal ischemia is induced by clamping the renal pedicles for 30 minutes. Sham surgery control consists of an identical procedure with the exception that there is no clamping of the renal pedicles. The compounds are administered 2 h post-induction of ischemia via jugular vein catheter. Animals are sacrificed at 24 h post-ischemia and renal function is determined by measurement of serum creatinine at 24 h post-ischemia. Blood is collected from the tail-vein of experimental, sham, and nonoperative control rats. Serum is isolated by centrifugation and stored with protease inhibitor. Serum creatinine is measured and reported in mg/dL. The variants TM-LE15 and TMD2-I424A are effective in suppressing renal injury as evidenced by reduction in SCr at 24 h post ischemia when compared to renal artery clamp (RAC) control.

TABLE 5

| Group | SCr (mg/dl) |
|---|---|
| RAC | 2.16 ± 0.27 |
| Sham Control | 0.23 ± 0.03 |
| RAC + TMD2-I424A | 0.68 ± 0.09 |
| RAC + TM-LE15 | 0.92 ± 0.13 |

SEQ ID NO: 1
MLGVLVLGALALAGLGFPAPAEPQPGGSQCVEHDCFALYPGPATFLNAS
QICDGLRGHLMTVRSSVAADVISLLLNGDGGVGRRRLWIGLQLPPGCGD
PKRLGPLRGFQWVTGDNNTSYSRWARLDLNGAPLCGPLCVAVSAAEATV
PSEIWEEQQCEVKADGFLCEFHFPATCRPLAVEPGAAAAAVSITYGTPF
AARGADFQALPVGSSAAVAPLGLQLMCTAPPGAVQGHWAREAPGAWDCS
VENGGCEHACNAIPGAPRCQCPAGAALQADGRSCTASATQSCNDLCEHF
CVPNPDQPGSYSCMCETGYRLAADQHRCEDVDDCILEPSPCPQRCVNTQ
GGFECHCYPNYDLVDGECVEPVDPCFRANCEYQCQPLNQTSYLCVCAEG
FAPIPHEPHRCQMFCNQTACPADCDPNTQASCECPEGYILDDGFICTDI
DNECENGGFCSGVCHNLPGTFECICGPDSALVRHIGTDCDSGKVDGGDS
GSGEPPPSPTPGSTLTPPAVGLVHSGLLIGISIASLCLVVALLALLCHL
RKKQGAARAKMEYKCAAPSKEVVLQHVRTERTPQRL
SEQ ID NO: 2
MLGVLVLGALALAGLGFPAPAEPQPGGSQCVEHDCFALYPGPATFLNAS
QICDGLRGHLMTVRSSVAADVISLLLNGDGGVGRRRLWIGLQLPPGCGD
PKRLGPLRGFQWVTGDNNTSYSRWARLDLNGAPLCGPLCVAVSAAEATV
PSEPIWEEQQCEVKADGFLCEFHFPATCRPLAVEPGAAAAAVSITYGTP
FAARGADFQALPVGSSAAVAPLGLQLMCTAPPGAVQGHWAREAPGAWDC
SVENGGCEHACNAIPGAPRCQCPAGAALQADGRSCTASATQSCNDLCEH
FCVPNPDQPGSYSCMCETGYRLAADQHRCEDVDDCILEPSPCPQRCVNT
QGGFECHCYPNYDLVDGECVEPVDPCFRANCEYQCQPLNQTSYLCVCAE
GFAPIPHEPHRCQMFCNQTACPADCDPNTQASCECPEGYILDDGFICTD
IDECENGGFCSGVCHNLPGTFECICGPDSALARHIGTDCDSGKVDGGDS
GSGEPPPSPTPGSTLTPPAVGLVHS
SEQ ID NO: 3
MLGVLVLGALALAGLGFPAPAEPQPGGSQCVEHDCFALYPGPATFLNAS
QICDGLRGHLMTVRSSVAADVISLLLNGDGGVGRRRLWIGLQLPPGCGD
PKRLGPLRGFQWVTGDNNTSYSRWARLDLNGAPLCGPLCVAVSAAEATV
PSEPIWEEQQCEVKADGFLCEFHFPATCRPLAVEPGAAAAAVSITYGTP
FAARGADFQALPVGSSAAVAPLGLQLMCTAPPGAVQGHWAREAPGAWDC
SVENGGCEHACNAIPGAPRCQCPAGAALQADGRSCTASATQSCNDLCEH
FCVPNPDQPGSYSCMCETGYRLAADQHRCEDVDDCILEPSPCPQRCVNT
QGGFECHCYPNYDLVDGECVEPVDPCFRANCEYQCQPLNQTSYLCVCAE
GFAPIPHEPHRCQMFCNQTACPADCDPNTQASCECPEGYILDDGFICTD
IDECENGGFCSGVCHNLPGTFECICGPDSALARHIGTDCDSGK
SEQ ID NO: 4
APAEPQPGGSQCVEHDCFALYPGPATFLNASQICDGLRGHLMTVRSSVA
ADVISLLLNGDGGVGRRRLWIGLQLPPGCGDPKRLGPLRGFQWVTGDNN
TSYSRWARLDLNGAPLCGPLCVAVSAAEATVPSEPIWEEQQCEVKADGF
LCEFHFPATCRPLAVEPGAAAAAVSITYGTPFAARGADFQALPVGSSAA
VAPLGLQLMCTAPPGAVQGHWAREAPGAWDCSVENGGCEHACNAIPGAP
RCQCPAGAALQADGRSCTASATQSCNDLCEHFCVPNPDQPGSYSCMCET
GYRLAADQHRCEDVDDCILEPSPCPQRCVNTQGGFECHCYPNYDLVDGE
CVEPVDPCFRANCEYQCQPLNQTSYLCVCAEGFAPIPHEPHRCQMFCNQ
TACPADCDPNTQASCECPEGYILDDGFICTDIDECENGGFCSGVCHNLP
GTFECICGPDSALARHIGTDCDSGKVDGGDSGSGEPPPSPTPGSTLTPP
AVGLVHS
SEQ ID NO: 5
APAEPQPGGSQCVEHDCFALYPGPATFLNASQICDGLRGHLMTVRSSVA
ADVISLLLNGDGGVGRRRLWIGLQLPPGCGDPKRLGPLRGFQWVTGDNN
TSYSRWARLDLNGAPLCGPLCVAVSAAEATVPSEPIWEEQQCEVKADGF
LCEFHFPATRPLAVEPGAAAAAVSITYGTPFAARGADFQALPVGSSAAV
APLGLQLMCTAPPGAVQGHWAREAPGAWDCSVENGGCEHACNAIPGAPR
CQCPAGAALQADGRSCTASATQSCNDLCEHFCVPNPDQPGSYSCMCETG
YRLAADQHRCEDVDDCILEPSPCPQRCVNTQGGFECHCYPNYDLVDGEC
VEPVDPCFRANCEYQCQPLNQTSYLCVCAEGFAPIPHEPHRCQMFCNQT
ACPADCDPNTQASCECPEGYILDDGFICTDIDECENGGFCSGVCHNLPG
TFECICGPDSALARHIGTDCDSGK
SEQ ID NO: 6
APAEPQPGGSQCVEHDCFALYPGPATFLNASQICDGLRGHLMTVRSSVA
ADVISLLLNGDGGVGRRRLWIGLQLPPGCGDPKRLGPLRGFQWVTGDNN
TSYSRWARLDLNGAPLCGPLCVAVSAAEATVPSEPIWEEQQCEVKADGF
LCEFHFPATCRPLAVEPGAAAAAVSITYGTPFAARGADFQALPVGSSAA
VAPLGLQLMCTAPPGAVQGHWAREAPGAWDCSVENGGCEHACNAIPGAP
RCQCPAGAALQADGRSCTASATQSCNDLCEHFCVPNPDQPGSYSCMCET
GYRLAADQHRCEDVDDCILEPSPCPQRCVNTQGGFECHCYPNYDLVDGE
CVEPVDPCFRANCEYQCQPLNQTSYLCVCAEGFAPIPHEPHRCQMFCNQ
TACPADCDPNTQASCECPEGYILDDGFICTDADECENGGFCSGVCHNLP
GTFECICGPDSALARHIGTDCDSGK
Human sTMD2 DNA sequence
SEQ ID NO: 7
GCCCCTGCCGAGCCTCAGCCTGGCGGCAGCCAGTGCGTGGAGCACGACT
GCTTCGCCCTGTACCCCGGACCCGCCACCTTCCTGAACGCCAGCCAGAT
CTGCGACGGCCTGCGGGGCCACCTGATGACCGTGCGGAGCAGCGTGGCC
GCCGACGTGATCAGCCTGCTGCTGAACGGCGACGGCGGCGTGGGCAGGC
GGAGGCTGTGGATCGGACTGCAGCTGCCCCCTGGCTGCGGCGACCCCAA
GAGGCTGGGCCCCCTGCGGGGCTTCCAGTGGGTGACCGGCGACAACAAC
ACCAGCTACAGCAGATGGGCCAGGCTGGACCTGAATGGCGCCCCTCTGT
GCGGCCCACTGTGCGTGGCCGTGTCTGCCGCCGAGGCCACCGTGCCCAG
CGAGCCCATCTGGGAGGAACAGCAGTGCGAAGTGAAGGCCGACGGCTTC
CTGTGCGAGTTCCACTTCCCCGCCACCTGCAGGCCTCTGGCCGTGGAAC
CTGGAGCCGCTGCTGCCGCCGTGAGCATCACCTACGGCACCCCCTTCGC
CGCCAGAGGCGCCGACTTCCAGGCCCTGCCCGTGGGCTCTTCTGCCGCC
GTGGCCCCCCTGGGGCTGCAGCTGATGTGCACCGCCCCTCCAGGCGCCG
TGCAGGGCCACTGGGCCAGAGAAGCCCCTGGCGCCTGGGACTGCAGCGT
GGAGAACGGCGGCTGCGAGCACGCCTGCAACGCCATCCCTGGCGCCCCT
AGGTGCCAGTGCCCTGCCGGAGCCGCCCTCCAGGCCGATGGCAGAAGCT
GCACCGCCAGCGCCACCCAGAGCTGCAACGACCTGTGCGAGCACTTCTG
CGTGCCCAACCCCGACCAGCCCGGCAGCTACAGCTGCATGTGCGAGACC
GGCTACCGGCTGGCCGCCGATCAGCACAGATGCGAGGACGTGGACGACT
GCATCCTGGAACCCAGCCCCTGCCCCCAGAGATGCGTGAACACCCAGGG
CGGCTTCGAGTGCCACTGCTACCCCAACTACGACCTGGTGGACGGCGAG
TGTGTGGAGCCCGTGGACCCCTGCTTCCGGGCCAACTGCGAGTACCAGT
GCCAGCCCCTGAACCAGACCAGCTACCTGTGCGTGTGCGCCGAAGGCTT
CGCCCCCATCCCCCACGAGCCCCACCGGTGCCAGATGTTCTGCAACCAG
ACCGCCTGCCCTGCCGACTGCGACCCCAATACCCAGGCCAGCTGCGAGT
GCCCCGAGGGCTACATCCTGGACGACGGCTTCATCTGCACCGACATCGA
CGAGTGCGAGAATGGCGGCTTCTGCAGCGGCGTGTGCCACAACCTGCCC
GGCACCTTCGAGTGCATCTGCGGCCCTGACAGCGCCCTGGCCCGGCACA
TCGGCACCGACTGCGATAGCGGCAAG
Human sTMD2-I424A DNA sequence
SEQ ID NO: 8
GCCCCTGCCGAGCCTCAGCCTGGCGGCAGCCAGTGCGTGGAGCACGACT
GCTTCGCCCTGTACCCCGGACCCGCCACCTTCCTGAACGCCAGCCAGAT
CTGCGACGGCCTGCGGGGCCACCTGATGACCGTGCGGAGCAGCGTGGCC
GCCGACGTGATCAGCCTGCTGCTGAACGGCGACGGCGGCGTGGGCAGGC
GGAGGCTGTGGATCGGACTGCAGCTGCCCCCTGGCTGCGGCGACCCCAA
GAGGCTGGGCCCCCTGCGGGGCTTCCAGTGGGTGACCGGCGACAACAAC
ACCAGCTACAGCAGATGGGCCAGGCTGGACCTGAATGGCGCCCCTCTGT
GCGGCCCACTGTGCGTGGCCGTGTCTGCCGCCGAGGCCACCGTGCCCAG
CGAGCCCATCTGGGAGGAACAGCAGTGCGAAGTGAAGGCCGACGGCTTC
CTGTGCGAGTTCCACTTCCCCGCCACCTGCAGGCCTCTGGCCGTGGAAC
CTGGAGCCGCTGCTGCCGCCGTGAGCATCACCTACGGCACCCCCTTCGC
CGCCAGAGGCGCCGACTTCCAGGCCCTGCCCGTGGGCTCTTCTGCCGCC
GTGGCCCCCCTGGGGCTGCAGCTGATGTGCACCGCCCCTCCAGGCGCCG
TGCAGGGCCACTGGGCCAGAGAAGCCCCTGGCGCCTGGGACTGCAGCGT
GGAGAACGGCGGCTGCGAGCACGCCTGCAACGCCATCCCTGGCGCCCCT
AGGTGCCAGTGCCCTGCCGGAGCCGCCCTCCAGGCCGATGGCAGAAGCT
GCACCGCCAGCGCCACCCAGAGCTGCAACGACCTGTGCGAGCACTTCTG
CGTGCCCAACCCCGACCAGCCCGGCAGCTACAGCTGCATGTGCGAGACC
GGCTACCGGCTGGCCGCCGATCAGCACAGATGCGAGGACGTGGACGACT
GCATCCTGGAACCCAGCCCCTGCCCCCAGAGATGCGTGAACACCCAGGG
CGGCTTCGAGTGCCACTGCTACCCCAACTACGACCTGGTGGACGGCGAG
TGTGTGGAGCCCGTGGACCCCTGCTTCCGGGCCAACTGCGAGTACCAGT
GCCAGCCCCTGAACCAGACCAGCTACCTGTGCGTGTGCGCCGAAGGCTT
CGCCCCCATCCCCCACGAGCCCCACCGGTGCCAGATGTTCTGCAACCAG
ACCGCCTGCCCTGCCGACTGCGACCCCAATACCCAGGCCAGCTGCGAGT
GCCCCGAGGGCTACATCCTGGACGACGGCTTCATCTGCACCGACGCCGA
CGAGTGCGAGAATGGCGGCTTCTGCAGCGGCGTGTGCCACAACCTGCCC
GGCACCTTCGAGTGCATCTGCGGCCCTGACAGCGCCCTGGCCCGGCACA
TCGGCACCGACTGCGATAGCGGCAAG
spTMD1 DNA Sequence
SEQ ID NO: 9
ATGCTGGGCGTGCTGGTGCTGGGAGCCCTGGCCCTGGCCGGCCTGGGCT
TTCCTGCCCCTGCCGAGCCTCAGCCTGGCGGCAGCCAGTGCGTGGAGCA
CGACTGCTTCGCCCTGTACCCCGGACCCGCCACCTTCCTGAACGCCAGC
CAGATCTGCGACGGCCTGCGGGGCCACCTGATGACCGTGCGGAGCAGCG
TGGCCGCCGACGTGATCAGCCTGCTGCTGAACGGCGACGGCGGCGTGGG
CAGGCGGAGGCTGTGGATCGGACTGCAGCTGCCCCCTGGCTGCGGCGAC
CCCAAGAGGCTGGGCCCCCTGCGGGGCTTCCAGTGGGTGACCGGCGACA
ACAACACCAGCTACAGCAGATGGGCCAGGCTGGACCTGAATGGCGCCCC
TCTGTGCGGCCCACTGTGCGTGGCCGTGTCTGCCGCCGAGGCCACCGTG
CCCAGCGAGCCCATCTGGGAGGAACAGCAGTGCGAAGTGAAGGCCGACG
GCTTCCTGTGCGAGTTCCACTTCCCCGCCACCTGCAGGCCTCTGGCCGT
GGAACCTGGAGCCGCTGCTGCCGCCGTGAGCATCACCTACGGCACCCCC
TTCGCCGCCAGAGGCGCCGACTTCCAGGCCCTGCCCGTGGGCTCTTCTG
CCGCCGTGGCCCCCCTGGGGCTGCAGCTGATGTGCACCGCCCCTCCAGG
CGCCGTGCAGGGCCACTGGGCCAGAGAAGCCCCTGGCGCCTGGGACTGC
AGCGTGGAGAACGGCGGCTGCGAGCACGCCTGCAACGCCATCCCTGGCG
CCCCTAGGTGCCAGTGCCCTGCCGGAGCCGCCCTCCAGGCCGATGGCAG
AAGCTGCACCGCCAGCGCCACCCAGAGCTGCAACGACCTGTGCGAGCAC
TTCTGCGTGCCCAACCCCGACCAGCCCGGCAGCTACAGCTGCATGTGCG
AGACCGGCTACCGGCTGGCCGCCGATCAGCACAGATGCGAGGACGTGGA
CGACTGCATCCTGGAACCCAGCCCCTGCCCCCAGAGATGCGTGAACACC
CAGGGCGGCTTCGAGTGCCACTGCTACCCCAACTACGACCTGGTGGACG
GCGAGTGTGTGGAGCCCGTGGACCCCTGCTTCCGGGCCAACTGCGAGTA
CCAGTGCCAGCCCCTGAACCAGACCAGCTACCTGTGCGTGTGCGCCGAA
GGCTTCGCCCCCATCCCCCACGAGCCCCACCGGTGCCAGATGTTCTGCA
ACCAGACCGCCTGCCCTGCCGACTGCGACCCCAATACCCAGGCCAGCTG
CGAGTGCCCCGAGGGCTACATCCTGGACGACGGCTTCATCTGCACCGAC
ATCGACGAGTGCGAGAATGGCGGCTTCTGCAGCGGCGTGTGCCACAACC
TGCCCGGCACCTTCGAGTGCATCTGCGGCCCTGACAGCGCCCTGGCCCG
GCACATCGGCACCGACTGCGATAGCGGCAAGGTGGACGGGGGCGACTCC
GGCTCCGGCGAGCCCCCTCCCAGCCCTACCCCCGGCAGCACCCTGACCC
CTCCCGCCGTGGGCCTGGTGCACAGC
spTMD1-I424A DNA Sequence
SEQ ID NO: 10
ATGCTGGGCGTGCTGGTGCTGGGAGCCCTGGCCCTCGCTGGACTGGGCT
TTCCTGCCCCTGCCGAGCCTCAGCCTGGCGGCAGCCAGTGCGTGGAGCA
CGACTGCTTCGCCCTGTACCCCGGACCCGCCACCTTCCTGAACGCCAGC
CAGATCTGCGACGGCCTGAGAGGCCACCTGATGACCGTGCGGAGCAGCG
TGGCCGCCGACGTGATCAGCCTGCTGCTGAACGGCGACGGCGGCGTGGG
CAGGCGGAGACTGTGGATCGGCCTGCAGCTGCCCCCTGGCTGCGGCGAC
CCCAAGAGACTGGGCCCCCTGCGGGGCTTCCAGTGGGTGACCGGCGACA
ACAACACCAGCTACAGCAGATGGGCCAGACTGGACCTGAATGGCGCCCC
TCTGTGCGGCCCACTGTGCGTGGCCGTGTCTGCTGCCGAGGCCACCGTG
CCCAGCGAGCCCATCTGGGAGGAACAGCAGTGCGAAGTGAAGGCCGACG
GCTTCCTGTGCGAGTTCCACTTCCCCGCCACCTGCAGACCCCTGGCCGT
GGAACCCGGCGCCGCTGCTGCAGCCGTGTCTATCACCTACGGCACCCCC
TTCGCCGCCAGAGGCGCCGACTTCCAGGCCCTGCCCGTGGGAAGCTCTG
CCGCCGTGGCCCCTCTGGGGCTGCAGCTGATGTGCACCGCCCCTCCAGG
CGCCGTGCAGGGCCACTGGGCCAGAGAAGCCCCTGGGGCCTGGGACTGC
AGCGTGGAGAACGGCGGCTGCGAGCACGCCTGCAACGCCATCCCTGGCG
CCCCTAGATGCCAGTGCCCTGCTGGAGCCGCCCTGCAGGCCGATGGCAG
AAGCTGCACCGCCAGCGCCACCCAGAGCTGCAACGACCTGTGCGAGCAC
TTCTGCGTGCCCAACCCCGACCAGCCTGGAAGCTACAGCTGCATGTGCG
AGACAGGCTACCGGCTGGCCGCCGATCAGCACAGATGCGAGGACGTGGA
CGACTGCATCCTGGAACCCAGCCCCTGCCCCCAGAGATGCGTGAACACC
CAGGGCGGCTTCGAGTGCCACTGCTACCCTAACTACGACCTGGTGGACG
GCGAGTGTGTGGAGCCCGTGGACCCCTGCTTCCGGGCCAACTGCGAGTA
CCAGTGCCAGCCCCTGAACCAGACCAGCTACCTGTGCGTGTGCGCCGAA
GGCTTCGCCCCCATCCCCCACGAGCCCCACCGGTGCCAGATGTTCTGCA
ACCAGACCGCCTGTCCTGCCGACTGCGACCCCAATACCCAGGCCAGCTG
TGAGTGCCCCGAGGGCTACATCCTGGACGACGGCTTCATCTGCACAGAC
GCCGACGAGTGCGAGAATGGCGGCTTCTGCAGCGGCGTGTGCCACAACC
TGCCCGGCACCTTCGAGTGCATCTGCGGCCCTGACAGCGCCCTGGCCAG
ACACATCGGCACCGACTGCGATAGCGGCAAGGTGGACGGGGGGGACGCC
GGAGCCGGCGAGCCTCCCCCCAGCCCTACCCCCGGCAGCACCCTGACCC
CTCCCGCCGTGGGCCTGGTGCACAGC
SEQ ID NO: 11
APAEPQPGGSQCVEHDCFALYPGPATFLNASQICDGLRGHLMTVRSSVA
ADVISLLLNGDGGVGRRRLWIGLQLPPGCGDPKRLGPLRGFQWVTGDNN
TSYSRWARLDLNGAPLCGPLCVAVSAAEATVPSEPIWEEQQCEVKADGF
LCEFHFPATCRPLAVEPGAAAAAVSITYGTPFAARGADFQALPVGSSAA
VAPLGLQLMCTAPPGAVQGHWAREAPGAWDCSVENGGCEHACNAIPGAP
RCQCPAGAALQADGRSCTASATQSCNDLCEHFCVPNPDQPGSYSCMCET
GYRLAADQHRCEDVDDCILEPSPCPQRCVNTQGGFECHCYPNYDLVDGE
CVEPVDPCFRANCEYQCQPLNQTSYLCVCAEGFAPIPHEPHRCQMFCNQ
TACPADCDPNTQASCECPEGYILDDGFICTDIDE
hsTM-LE15 DNA Sequence
SEQ ID NO: 12
GCCCCTGCCGAGCCTCAGCCTGGCGGCAGCCAGTGCGTGGAGCACGACT
GCTTCGCCCTGTACCCCGGACCCGCCACCTTCCTGAACGCCAGCCAGAT
CTGCGACGGCCTGCGGGGCCACCTGATGACCGTGCGGAGCAGCGTGGCC
GCCGACGTGATCAGCCTGCTGCTGAACGGCGACGGCGGCGTGGGCAGGC
GGAGGCTGTGGATCGGACTGCAGCTGCCCCCTGGCTGCGGCGACCCCAA
GAGGCTGGGCCCCCTGCGGGGCTTCCAGTGGGTGACCGGCGACAACAAC
ACCAGCTACAGCAGATGGGCCAGGCTGGACCTGAATGGCGCCCCTCTGT
GCGGCCCACTGTGCGTGGCCGTGTCTGCCGCCGAGGCCACCGTGCCCAG
CGAGCCCATCTGGGAGGAACAGCAGTGCGAAGTGAAGGCCGACGGCTTC
CTGTGCGAGTTCCACTTCCCCGCCACCTGCAGGCCTCTGGCCGTGGAAC
CTGGAGCCGCTGCTGCCGCCGTGAGCATCACCTACGGCACCCCCTTCGC
CGCCAGAGGCGCCGACTTCCAGGCCCTGCCCGTGGGCTCTTCTGCCGCC
GTGGCCCCCCTGGGGCTGCAGCTGATGTGCACCGCCCCTCCAGGCGCCG
TGCAGGGCCACTGGGCCAGAGAAGCCCCTGGCGCCTGGGACTGCAGCGT
GGAGAACGGCGGCTGCGAGCACGCCTGCAACGCCATCCCTGGCGCCCCT
AGGTGCCAGTGCCCTGCCGGAGCCGCCCTCCAGGCCGATGGCAGAAGCT
GCACCGCCAGCGCCACCCAGAGCTGCAACGACCTGTGCGAGCACTTCTG
CGTGCCCAACCCCGACCAGCCCGGCAGCTACAGCTGCATGTGCGAGACC
GGCTACCGGCTGGCCGCCGATCAGCACAGATGCGAGGACGTGGACGACT
GCATCCTGGAACCCAGCCCCTGCCCCCAGAGATGCGTGAACACCCAGGG
CGGCTTCGAGTGCCACTGCTACCCCAACTACGACCTGGTGGACGGCGAG
TGTGTGGAGCCCGTGGACCCCTGCTTCCGGGCCAACTGCGAGTACCAGT
GCCAGCCCCTGAACCAGACCAGCTACCTGTGCGTGTGCGCCGAAGGCTT
CGCCCCCATCCCCCACGAGCCCCACCGGTGCCAGATGTTCTGCAACCAG
ACCGCCTGCCCTGCCGACTGCGACCCCAATACCCAGGCCAGCTGCGAGT
GCCCCGAGGGCTACATCCTGGACGACGGCTTCATCTGCACCGACATCGA
CGAG
SEQ ID NO: 13
MLGVLVLGALALAGLGFP

We claim:

1. A method of treating AKI in a patient in need thereof which comprises administering to the patient an sTM variant that does not bind thrombin, wherein the sTM variant consists of the amino acid sequence of SEQ ID NO: 6 or SEQ ID NO: 11.

2. A method of preventing AKI in a patient susceptible to AKI comprising administering to the patient an sTM variant that does not bind thrombin, wherein the sTM variant consists of the amino acid sequence of SEQ ID NO: 6 or SEQ ID NO: 11.

3. The method of claim 1, wherein the variant has a Kd value of >4300 for thrombin binding under the BIAcore assay conditions described in Example 3.

4. (canceled)

5. An sTM variant consisting of the amino acid sequence shown in SEQ ID NO: 11.

6. A pharmaceutical composition comprising the sTM variant of claim 5 and a pharmaceutically acceptable excipient.

7-11. (canceled)
